(19) United States
(12) Patent Application Publication
(10) Pub. No.: US 2008/0076111 A1
(43) Pub. Date: Mar. 27, 2008

(54) Methods and kits for determining sperm cell number

(75) Inventor: Shafrira Shai, Kiryat-Haim (IL)

Correspondence Address: Martin D. Moynihan;PRTSI, Inc., P.O. Box 16446, Arlington, VA, 22215, US

(73) Assignee: Dyn-BioShaf (2006) Ltd., Caesarea (IL)

(21) Appl. No.: 11/524,947

(22) Filed: Sep. 22, 2006

(51) Int. Cl. C12Q 1/68; C12Q 1/00; G01N 33/567

(52) U.S. Cl. 435/4; 435/6; 435/7.21

(57) ABSTRACT

A flow cytometry method and kit is provided for quantifying an absolute number of mature sperm cells in a semen sample, the method comprising (a) determining a total particle number (T); (b) determining a number of DNA comprising particles (D); (c) determining a number of cells which are not mature sperm cells (B) and; (d) determining a background signal (A); wherein steps (a) to (d) are not effected in a single aliquot of the semen sample, and whereas the number of mature sperm cells in the semen sample is equal to [(T×D)/100]−(A+B), thereby quantifying the absolute number of mature sperm cells in the semen sample.

### FIELD AND BACKGROUND OF THE INVENTION

The present invention relates to a flow cytometry method for determining sperm cell number.

Infertility affects 10-18% of couples in industrially developed countries and it is likely that the global deterioration of semen parameters (most pronounced in industrialized societies), will increase these percentages.

Semen analysis is a crucial, primary test for both identifying male infertility and determining its causes. Assessment of sperm concentration is also an important procedure for evaluating effects of drugs on testis function in laboratory animals. Conventional semen analysis uses quantitative as well as qualitative parameters. Traditionally, laboratory experts perform semen analysis using a light microscope, with either a fresh or stained sample. Due to the nature of the assay there is a high percentage of inter- and intra-laboratory variability for both the evaluation of morphology and in the assessment of quantitative parameters such as sperm concentration [Matson, P. L., Hum Reprod 1995; 10:620-625]. Human observers typically assess a few hundred cells in a population of millions, which may lead to an incorrect reflection of the whole sample. In addition, human evaluation is subjective and depends on the experience and competency of the observer.

Application of a rapid, automated and precise method for the evaluation of both semen quality and quantity is therefore advantageous in many aspects. In the last three decades several techniques were introduced in an effort to obtain objective results for the traditionally evaluated parameters such as motility, morphology and cell concentration.

Recently, flow cytometry technology has been used to study diagnostic parameters such as reproductive capacity. The Flow Cytometer (FC) has been developed such that it is cost-effective for diagnostic functions; with an output far exceeding that obtained using a microscope. Flow cytometry offers speed, accuracy, precision and a capacity to assess large cells (such as immature sperm cells and WBCs) that may be overlooked using other techniques. Recently, flow cytometry has been applied to evaluate sperm cell membrane integrity, mitochondrial function, acrosomal status, chromatin structure, cation concentrations, sperm-bound antibodies and chromosomal analysis.

At present, flow cytometry methods for sperm cell counting are based on counting events which enter a defined gate “sperm window”. Sperm cell size is analyzed by forward and side angle light scatter and viability is analyzed by fluorescent staining reagents for DNA.

Christensen et al. [J. Andrology, 2004 25(2):255-64] teach a simple gate analysis of sperm cell concentration and viability using fluorescent beads. The method is mainly used for analyzing high sperm concentration (boar, bull). As indicated optimal results are obtained when the sperm concentration is between 105-106 per ml.

Evenson et al. [J. Dairy Sci., 1993, 76:86-94] teach a traditional flow cytometry method for determining sperm cell concentration in (bovine) semen samples. The method is based on staining the sample with a DNA stain and mixing the same sample with a known concentration of fluorescent beads. The sperm count is determined based on the ratio of fluorescent beads to sperm cells. Sperm concentration is then determined based on previously determined bead concentration and the sperm to bead ratio. Evenson et al does not teach white blood cell analysis, size analysis and background analysis for the determination of cell count.

Ferrara et al [Clin. Chem. 1997:43:5, 801-807] and Shai et al [Fertil Steril 2005; 83:1034-38] both teach analysis of sperm using flow cytometry in a single test tube with the aid of fluorescent beads. The analyses are effected in gated studies where semen qualification reagents are also added into the same tube. Such methods are inherently limited since the analysis of one parameter typically affects the analysis of a second parameter. For example, Ferrara et al reported that in his method the high fluorescence background of sperm cells interfere with the analysis of white blood cells. None of the methods described hereinabove relate to background signal.

There is thus a widely recognized need for, and it would be highly advantageous to have highly accurate flow cytometry based methods, to calculate sperm cell number in biological samples.

### SUMMARY OF THE INVENTION

According to one aspect of the present invention there is provided a flow cytometry method for quantifying an absolute number of mature sperm cells in a semen sample, the method comprising:

(a) determining a total particle number (T);

(b) determining a number of DNA comprising particles (D);

(c) determining a number of cells which are not mature sperm cells (B); and

(d) determining a background signal (A);

wherein steps (a) to (d) are not effected in a single aliquot of the semen sample, and whereas the number of mature sperm cells in the semen sample is equal [(T×D)/100]−(A+B), thereby quantifying the absolute number of mature sperm cells in the semen sample.

According to another aspect of the present invention there is provided a flow cytometry method for quantifying a relative number of mature sperm cells in a semen sample, the method comprising:

(a) determining a number of DNA comprising particles (D);

(b) determining a number of cells which are not mature sperm cells (B); and

(c) determining a background signal (A);

wherein steps (a)-(c) are not effected in a single aliquot of the semen sample, and whereas the number of mature sperm cells in the semen sample is equal to D−(A+B), thereby quantifying the relative number of mature sperm cells in the semen sample.

According to yet another aspect of the present invention there is provided a kit for determining a number of sperm cells in a semen sample, wherein the kit comprises:
(i) a white blood cell specific antibody;
(ii) a DNA staining agent;
(iii) a plurality of fluorescent beads; and
(iv) a plurality of tubes wherein each four of the tubes are packaged in a single packaging.

According to further features in preferred embodiments of the invention described below, the number of cells which are not mature sperm cells comprises a white blood cell number, and/or a number of cells of a substantially different size than the mature sperm cells.

According to still further features in the described preferred embodiments the aliquots comprise an identical volume.

According to still further features in the described preferred embodiments each of the background signal, the total particle number, the number of DNA comprising particles and the number of cells which are not mature sperm cells is determined in a separate aliquot.

According to still further features in the described preferred embodiments the determining the white blood cell number is effected via a white blood cell specific antibody.

According to still further features in the described preferred embodiments the white blood cell specific antibody is anti-CD-45.

According to still further features in the described preferred embodiments step (a) is effected via fluorescent beads.

According to still further features in the described preferred embodiments the determining the number of DNA comprising particles is effected via a DNA staining agent.

According to still further features in the described preferred embodiments the DNA staining agent is acridine orange or propidium iodide.

According to still further features in the described preferred embodiments the semen sample is a human semen sample.

According to still further features in the described preferred embodiments the semen sample is a bull semen sample.

The present invention successfully addresses the shortcomings of the presently known configurations by providing an accurate and reproducible method for counting sperm cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-F are graphs depicting the reading frame for the flow cytometry method of the present invention. FIG. 1A is a graph of forward scatter/side scatter (FSC/SSC) and depicts the “cells window”. FIGS. 1B-D are graphs depicting the fluorescence level for the green light (FL1)—RN2, the orange light (FL2)—RN4 and the red light (FL3)—RN6 respectively. FIG. 1E is a graph of FL1/FSC and depicts windows based on size and fluorescence level of the green light—this graph is used to identify WBC and round cells. FIG. 1F is a graph of FSC/FL3 and depicts “cell window”—R3 and “beads window”—R4.

### DESCRIPTION OF THE PREFERRED EMBODIMENTS

The present invention is of a flow cytometry method and kit which can be used for obtaining an accurate sperm count from a semen sample.

The principles and operation of the flow cytometry method according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Semen analysis is usually performed with the aid of manual techniques and microscopic evaluation by laboratory operators. The test is tedious, expensive and also not standardized: it is affected by a wide imprecision related to high variability of microscopic evaluation, influencing its clinical validity.

The use of flow cytometry for sperm analysis is an attempt to address the long-standing problem of the subjective nature of the manual method commonly used to perform semen samples analysis. Other sources of laboratory variation arise from the number of sperm cell analyzed, usually lower for manual techniques. Because of time and cost restraints, most laboratories analyze only 50 to 100 sperm to compute the percentage of each cell population and the viability rate: this small sample from a population of millions probably results in a statistical sampling error. Flow cytometry, on the other hand permits the analysis of many millions of cells in few seconds.

At present, flow cytometry methods for sperm cell counting are based on counting events which enter a defined gate “sperm window”. Sperm cell size is analyzed by forward and side angle light scatter and viability is analyzed by fluorescent staining reagents for DNA.

Evenson et al. [J. Dairy Sci., 1993, 76:86-94] teach a traditional flow cytometry method for determining bull sperm concentration using acridine orange or propidium iodide (PI) DNA staining and fluorospheres. This method is not appropriate for analyzing human samples, since it does not take into account the cell heterogeneity of human semen (e.g., immature germ cells, blood cells, epithelial cells, and cellular debris) in addition to spermatozoa.

Ferrara et al [Clin. Chem. 1997:43:5, 801-807] and Shai et al [Fertil Steril 2005; 83:1034-38] both teach analysis of human sperm using flow cytometry in a single test tube with the aid of fluorescent beads. The analyses are effected in gated studies where semen qualification reagents are also added to the same tube. Such methods are inherently limited since the analysis of one parameter typically affects the analysis of a second parameter. For example, Ferrara et al reported that in his method the high fluorescence background of sperm cells interfere with the analysis of white blood cells. None of the methods described hereinabove relate to background signal.

Whilst reducing the present invention to practice, the present inventors have discovered that subtracting a non sperm cell number from a total cell number which has been adjusted to take into account the background signal greatly increases the accuracy of the sperm counting method. Furthermore, by measuring the non-sperm cell number, background signal and total cell number each in separate aliquots of the same semen sample, the problem of “contaminating particles” entering defined windows is avoided. This method was shown to be accurate for samples with low sperm counts (Example 2) and highly reproducible as detailed in Tables 4 and 5 of Example 3 hereinbelow.

Thus, according to one aspect of the present invention, there is provided a flow cytometry method for quantifying an absolute number of mature sperm cells in a semen sample, the method comprising:

(a) determining a total particle number (T);

(b) determining a number of DNA comprising particles (D);

(c) determining a number of cells which are not mature sperm cells (B); and

(d) determining a background signal (A);

wherein steps (a) to (d) are not effected in a single aliquot of the semen sample, and whereas the number of mature sperm cells in the semen sample is equal [(T×D)/100]−(A+B), thereby quantifying the absolute number of mature sperm cells in the semen sample.

As used herein, the phrase “mature sperm cells” refers to non-round sperm cells. Since mature sperm cells typically comprise head body and tail, they are no longer round. Preferably, the mature sperm cells are viable and motile.

The semen sample may be obtained from the ejaculate of any male mammal. According to a preferred embodiment of this aspect of the present invention the mammal is a man or a bull.

As semen samples are relatively viscous, it is normally preferred that the semen sample is diluted. The dilution of the sample is preferably performed using a diluent which sustains viability and motility of the sperm cells during the determination, such as phosphate-buffered saline with nutrient mixture. In order to obtain an even higher degree of accuracy, automated dilution of the semen may be applied. Thereby, the operator-dependant part of the process is eliminated which makes the process highly reproducible.

The method of the present invention is performed using a Flow Cytometer, such as a laser scanning Cytometer. A Flow Cytometer typically consists of a laser light source, flow measurement chamber, and an optical system consisting of lenses, filters, and light detectors. Two photo-multiplier tubes (light detectors), one at 180 degrees and one at 90 degrees to the laser, are used to measure forward and right-angle scatter, respectively. Three fluorescence detectors, each consisting of a filter and photomultiplier tube, are used to detect fluorescence. The three detectors sense green, orange, and red fluorescence. Cells are identified by sort logic applied to all five of the detector signals using a computer.

Exemplary flow Cytometers that may be used in this aspect of the present invention are manufactured by companies such as Becton Dickinson (USA), Backman Coulter (USA), Partec (Germany) with the following specifications:

Laser excitation of 488 nm (blue laser, Argon laser)

Forward light Scatter FSC

Side light Scatter SSC

FL1—green filter—520 nm

FL2—orange filter—560 nm

FL3—red filter—660 nm.

Exemplary software that may be used to analyze the flow cytometry results include, but is not limited to those manufactured by:

Becton Dickinson—for instruments such as FacScan, FACSCalibur-CellQuest software,

Beckman Coulter—instruments such as Epics XL, and Cytomics FC 500,—Expo-32 or CXP software

Partec—instruments such as PAS and CyFlow—FloMax software

A typical analysis of semen may be performed by first measuring the right-angle and forward light scatter of the cells. The resulting scatter graph is used to identify the counting gate, a set of parameters used to select a subpopulation of cells for further study. The gated area of the scatter graph is the portion in which the cells of interest are found. The gate parameters are selected so that only this cell subpopulation is reported in subsequent fluorescence studies.

As mentioned hereinabove and further described below, the method of the present invention is based on determining a total cell number which has been adjusted to take into account the background signal and subtracting there from irrelevant events comprising the number of cells which are not mature sperm cells.

The algorithm for performing such a function may be incorporated into a software package or written on a C.D. such that the information obtained from the Flow Cytometer may be directly converted into a total sperm cell number.

In order to obtain an absolute total cell number, an internal concentration standard is used to obtain an absolute number of particles in the semen sample. Multiplication of this number by the number of cells in the sample, as further described hereinbelow while taking into account the background signal calculates the absolute total cell number. The internal concentration standard may be any concentration standard that can be suitably mixed with the sample (in a ratio of 1:1) and detected by the flow Cytometer within the reading time to function as a reference indicative of the concentration of the sperm cells. The internal concentration standard may suitably be constituted by standardization particles, the standardization particles being added in a predetermined number per weight or normally volume amount of the sample. For example, the standardization particles may be fluorescent particles, more preferably fluorescent beads, having a fluorescent quality distinguishable from the fluorescent qualities of the sperm cells, but of a similar size to the sperm cells. Exemplary fluorescent beads which may be used according to this aspect of the present invention are red fluorescent beads commercially available from Polysciences Inc, Washington, Pa.

The total number of fluorescent beads in a known volume of buffer (e.g. 0.5 ml) is known and the dilution of semen sample is also known. The unknown number of cells in an equal volume of diluted semen sample (e.g. 0.5 ml) is determined by dividing the counted number of beads by the counted number of cells and then multiplying by the dilution factor and by the number of beads in 0.5 ml. The beads may be provided in a suspension comprising beads and diluent. It is an advantage of a suspension comprising both the beads and the diluent that the suspension may be manufactured by a manufacturer in a highly automated process to obtain a very accurate number of beads in the suspension. Furthermore, the suspension comprising the diluent and the beads may be manufactured in tubes, the tubes being suitable for measuring chambers in fluorescent activated cell sorters, such as Flow Cytometers. Thereby, inaccuracies originating from redistribution and dilution of the suspension are minimized.

Preferably the diluent comprises a medium capable of preventing sperm cells from sticking to the side walls of the measuring chamber or measuring tube, e.g. a chemical compound, such as a protein, (e.g. BSA), or another suitable compound such as polyvinyl alcohol (PVA).

The total number of particles in a defined window may be calculated as described in the Examples section below and further described by Eustache et al [F. Eustache et al, Journal of Andrology, Vol 22, Issue 4 558-567, 2001]. Typically, the total cell count window measures particles of a size similar to sperm cells (e.g. greater than 3 and less than 10 microns).

As mentioned above, in order to calculate an absolute total cell number, the percentage of particles that comprise DNA must be determined. According to one embodiment of this aspect of the present invention this may be effected by contacting an aliquot of the sample with a DNA staining agent.

Following a suitable length of time, the cells are typically rinsed from excess staining agent and the percentage of stained cells in the sample aliquot is measured by flow cytometry. The sample is typically contacted with the DNA staining agent for a sufficient time such that following washing, substantially all cells are detected as being detected by the flow Cytometer (e.g. 30 minutes at room temperature).

Exemplary DNA staining agents include but are not limited to acridine orange and propidium iodide.

According to this aspect of the present invention, the final total cell number is calculated following reduction of the background signal, so as to take into account the relevant auto-fluorescence background.

Measurement of the background signal is effected by measuring the fluorescent signal from the cells of a known volume of semen sample by flow Cytometry.

As mentioned hereinabove, in order to determine a mature sperm cell count, the number of cells that are not mature sperm cells are subtracted from the background-adjusted, total cell count. According to this aspect of the present invention, the cells that are not mature sperm cells comprise white blood cells and cells of a substantially different size and shape to mature sperm cells.

Detection of white blood cells by FACS is typically effected via white blood cell specific antibodies.

As used herein, the phrase “white blood cell specific antibodies” refers to antibodies that preferentially bind to white blood cells as opposed to other cells in the semen sample. Typically, the white blood cell specific antibodies of the present invention are monoclonal and have an affinity at least ten times greater for white blood cells than stem cells.

White blood cells specific monoclonal antibodies are widely available. Examples of white blood cell specific antibodies include but are not limited to mouse anti-human CD-45 and anti-human CD-53. Preferably, the monoclonal antibody is antihuman-CD-45 as this is particularly selective and specific for human white blood cells.

As mentioned hereinabove, cells that are not mature sperm cells also comprise cells of a substantially different size and shape to mature sperm cells. As sperm cells mature, they form a tail and their cytoplasm contracts. Thus, immature sperm cells may be distinguished from mature sperm cells by their size and shape. Determination of the number of cells in a semen sample which are of a different size and shape to that of mature sperm cells (i.e. immature sperm cells) may be effected by analyzing the cells using a gate specified therefore as further described in the Examples section hereinbelow.

The method of the present invention is effected such that the measured parameters described hereinabove are not effected in a single aliquot of the sample. Preferably, each measured parameter is effected in an individual aliquot such that the analysis of one parameter does not affect the analysis of a second parameter. Preferably, each aliquot comprises an identical volume so that cell numbers may be easily calculated.

Preferably, the measurements are effected on the same day and more preferably simultaneously so as to minimize any external effect on the cell population of the semen sample. In addition, it is preferable that the aliquots are treated in identical fashions, such that if one aliquot of the sample is for example diluted with specific Buffer the other aliquot is diluted in the same manner.

It will be appreciated that the method of the present invention may also be used to determine a relative number of sperm cells in a semen sample. The method is effected in an identical fashion to that described hereinabove except a total particle number is not measured.

The present invention also contemplates analyzing other sperm cell parameters such as sperm motility based on the assay of the present invention. Thus, the present invention may also be used for determining a number of cells in a semen sample that are motile and/or viable. An exemplary agent for staining motile sperm cells is rhodamine. Agents that selectively stain non-viable sperm cells are generally capable of entering cells only through a leaking or defective plasma membrane. Typical DNA staining agent capable of staining dying cells include, propidium iodide, PI and ethidium-homodimer-2, EHD2.

The methods of the present invention may be used for the routine evaluation of semen, e.g. for artificial insemination and for determination of the degree of dilution required for securing an adequate number of sperm cells in each insemination dose.

The method of the present invention may also be used to determine the absolute number of sperm cells produced by animals subjected to toxicology or other pharmacological experiments. Furthermore, the method of the present invention may be used to prove that animals or human beings have been influenced by e.g. a toxicological environment or chemical invasions into the body.

It will be further appreciated that the method of the present invention may also be used to determine a cell population number in a semen sample other than sperm cells. Thus, for example the method may be used to determine a white blood cell number.

Determination of white blood cell count in semen samples may have various advantages. High concentrations of WBC's (leucocytospermia) may indicate the presence of an infection in the reproductive tract. Furthermore, high concentrations of these cells can have detrimental effects on the semen, including reduction in volume, sperm density, motility, and forward progression. WBC's can also impair sperm function as a result of the various reactive oxygen species they may release, along with possible secretions of cytotoxic cytokines.

Thus, in parallel (or alternatively) to the method described above for ascertaining sperm cell number, measurement of white blood cell number would be effected by subtracting from the total cell number, the number of cells that are not white blood cells (e.g. sperm cells), wherein the measurements are performed in more than one aliquot.

The components used to analyze semen samples may be provided as part of a kit.

Thus, according to another aspect of the present invention there is provided a kit for determining a number of sperm cells in a semen sample, wherein the kit comprises:
(i) a white blood cell specific antibody;
(ii) a DNA staining agent;
(iii) a plurality of fluorescent beads; and
(iv) a plurality of tubes wherein each four of said tubes are packaged in a single packaging.

The kit of the present invention may, if desired, be presented in a pack which may contain one or more units of the kit of the present invention. The pack may be accompanied by instructions for using the kit explaining that each cell parameter to be measured is effected in a separate tube.

As used herein, the term tube refers to any container which may be used to analyze cells in a flow Cytometer. The tube may be part of a multi-wells plate, each well representing one tube For example, CD4/3 and CD813 tubes manufactured by Becton Dickinson (EP 0 568 183) containing a predetermined number of beads in a diluent may be provided in the kit. Furthermore, tubes containing only beads, such as “TruCount” tubes manufactured by Becton Dickinson and containing a predetermined number of beads, may also be used.

The pack may also be accommodated by a notice associated with the container in a form prescribed by a governmental agency regulating the manufacture, use or sale of laboratory supplements, which notice is reflective of approval by the agency of the form of the compositions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Comparison Between Traditional Cell Counting Method and the Flow Cytometry Method of the Present Invention

Materials and Methods

Traditional sperm cell counting: Sperm cells and WBCs were counted from two semen samples by a professional laboratory technician. The counting was performed using a light microscope, slides and a specific stain according to the latest manual of the W.H.O [World Health Organization. Laboratory manual for the examination of human semen and sperm-cervical mucus interaction. 4^{th} ed. New York: Cambridge University Press, 1999]. The counting procedure was repeated three times for both semen samples and the mean result was recorded.

Flow cytometry sperm cell counting: Sperm cells were counted using four separate tubes:

Tube a=background;

Tube b=white blood cells;

Tube c=DNA; and

Tube d=Total cell number

The semen samples were diluted 20 fold in Assay buffer (PBS/BSA). Specifically, 0.5 ml of sample was diluted with 9.5 ml of assay buffer. 1 ml of diluted sample was transferred to tubes 1-3. 0.5 ml of diluted sample was transferred to tube 4. 0.01 ml of monoclonal anti Human CD-45 labeled with FITC (IQ Products, Groningen, the Netherlands) was added to tube b. 0.01 ml of Acridine Orange was added to tube c. All 4 tubes were incubated at room temperature (RT) for 30 minutes.

Tubes a-c were washed (tube d was left as is—i.e. without washing) with 1 ml of Assay Buffer, followed by vortexing and centrifuging at 250 g for 5 minutes. The supernatants were removed and an additional washing step was applied as before. Pellets were re-suspended in 1 ml of Assay Buffer. 0.5 ml of red fluorescent beads (Polysciences Inc, Washington, Pa.) were added to tube 4.

All 4 tubes were analyzed using 3 different Flowcytometers, as follows:
1. FACScan, FacsCalibure—Becton Dickinson Immunocytometry System, 2350 Qume Drive San Jose, Calif. 95131
2. Epics XL, and Cytomics FC 500—Beckman Coulter, Inc—4300 N. Harbor Boulevard P.O. Box 3100 Fullerton, Calif. 92834-3100 USA
3. PAS, CyFlow—Partec GmBh, Otto-Hahn-Straffe, 32 D-48161 Munster, Germany.

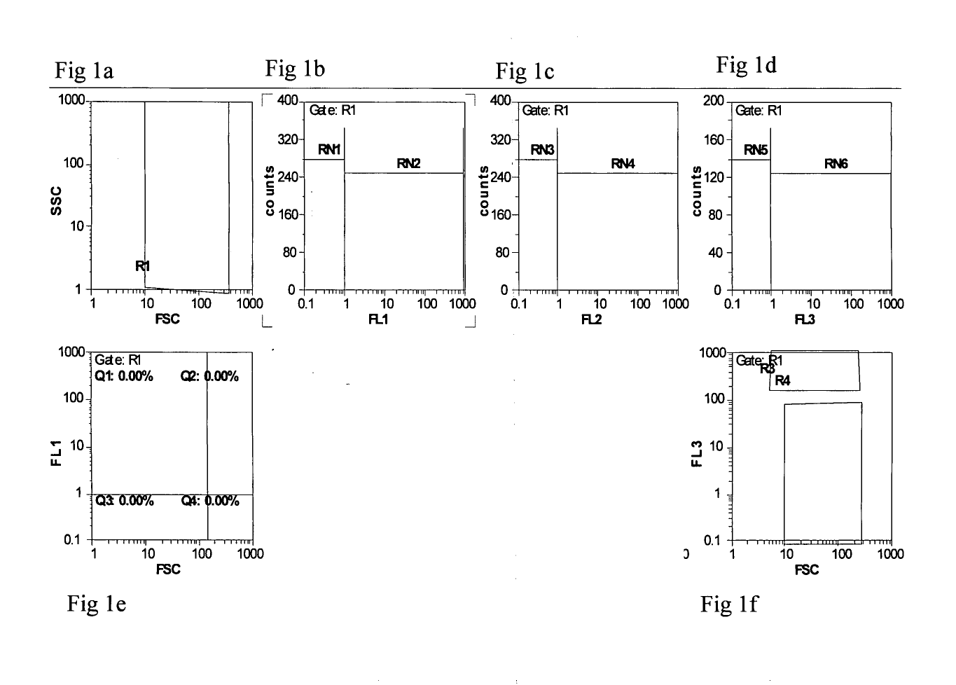

The tubes were analyzed using a pre-designed reading frame (FIGS. 1A-F). The Flowcytometer was supplied with an argon laser (488 nm), FSC (forward scatter), SSC (side scatter) FL1 (green light 520 nm) FL2 (orange light 560 nm) and FL3 (red light 600 nm).

Calculation of sperm cell count: First, a total count of all events that are >3 microns and <10 microns in the diluted semen sample was determined by reading tube d. This count includes counting events in gate R3 from not only sperm cells, but also other kind of cells such as WBC (white blood cells), ISC (immature sperm cells) and non-cells aggregates and dirties and counting events in gate R4 from the fluorescent beads (events from the known number of red fluorescent beads in the tube)

Accordingly, Total Count=R4/R3×dilution factor×total number of fluorescence beads in 0.5 ml×2=total cells count/ml.

This calculation is based on using known number of fluorescence beads (150000) in 0.5 ml solution [F. Eustache et al., Journal of Andrology, Vol 22, Issue 4, 558-567, 2001].

Second, from the total count, the percent of events that contain DNA (RN6) was converted to number of cells (tube c). The background noise was reduced (tube a-RN6).

Accordingly, total number of cells in sample/ml={RN6(tube c)−RN6(tube a)}×total cell count/100.

Thirdly, the WBC count parameter was evaluated by measuring the fluorescence level obtained from reading tube b. The Flowcytometer counts the fluorescent cells, which are gated in Q1 & Q2 (FSC/FL1). The background noise was reduced (tube 1 control) from the fluorescent cell count and the correct number of WBCs was calculated. Using this method, aggregates and dirties did not interfere with the counting result because counting events which were not stained by DNA specific staining reagent were subtracted from the total cell count. The relevant auto-fluorescence background was subtracted and therefore did not interfere with the final result.

Accordingly, number of white blood cells/ml={Q1(1d)+Q2(1d)}−

{Q1(1a)+Q2(1a)}×cells in sample/100.

Fourthly, cells that were larger than sperm cells and WBCs that were not labeled by the WBC specific monoclonal antibody (such as Immature sperm cells) were also obtained from reading tube b.

Accordingly, number of immature sperm cells/ml=Q4(1d)×cells in

sample/100.

Number of Sperm cells: cells in sample−(WBC+immature sperm cells).

Results

Sample 1:
A. Total count: R4/R3×dilution factor×beads 0.5 ml×1=total cells count/ml

8634/1408×20×150000×2=36.7×10⁶ cell/ml
B. Number of Cells in sample: {RN6(tube c−RN6(tube a)}×total cell count/100=cells in sample/ml

(90.13−6.82)×36.7 10⁶/100=30.610⁶ cell/ml
C. Number of WBCs: {Q1(1d)+Q2(1d)}−{Q1(1a)+Q2(1a)}*cells in sample/100

(6.44+3.97)−(5.16+3.05)=2.2×30.6×10⁶/100=0.67×10⁶ cell/ml
D. Number of ISC and other round cells (not WBC): Q4(1d)*cells in sample/100=immature sperm cells/ml

2.35×30.6×10⁶/100=0.71×10⁶ cell/ml
E. Number of sperm cells: cells in sample−(WBC+Round cells)

30.6×10⁶−(0.67×10⁶+0.71×10⁶)=29.2×10⁶ cell/ml

Sample 2:
A. Total count: R4/R3×dilution factor×beads 0.5 ml×2=total cells count/ml

7293/2697×20×150000×2=16.210⁶ cell/ml
B. Number of Cells in sample: {RN6(tube c−RN6(tube a)}×total cell count/100=cells in sample/ml

(95.31−6.12)×16.210⁶/100=14.410⁶ cell/ml
C. Number of WBCs: {Q1(1d)+Q2(1d)}−{Q1(1a)+Q2(1a)}*cells in sample/100

(11.97+0.37)−(6.44+0.28)=5.62×14.410⁶/100=0.8×10⁶ cell/ml
D. Number of ISC and other round cells (not WBC): Q4(1d)*cells in sample/100=immature sperm cells/ml

0.08×14.410⁶/100=0.0110⁶ cell/ml
E. Number of sperm cells: cells in sample−(WBC+Round cells)

14.410⁶−(0.8×10⁶+0.01×10⁶)=13.6×10⁶ cell/ml

Table 1 below summarizes the sperm count result by the routine method that was performed by a highly professional laboratory technician and by the Flowcytometric based method:

TABLE 1

| Sperm Count | Sample 1 | Sample 2 |
|---|---|---|
| Routine method | 30 × 10⁶ cell/ml | 12.8 × 10⁶ cell/ml |
| Flow cytometry method | 29.2 × 10⁶ cell/ml | 13.6 × 10⁶ cell/ml |

Table 2 below summarizes the WBC count result by the routine method and by the Flowcytometric based method:

TABLE 2

| Sperm Count | Sample 1 | Sample 2 |
|---|---|---|
| Routine method | 0.5 × 10⁶ cell/ml | 0.8 × 10⁶ cell/ml |
| Flow cytometry method | 0.67 × 10⁶ cell/ml | 0.8 × 10⁶ cell/ml |

The number of sperm cells in samples 1 and 2 were calculated without reduction of background noise and aggregates for Tables 1 and 2.

Next, the number of sperm cells in samples 1 and 2 were calculated with reduction of background noise and aggregates:

Sample 1:

Total cells count=36.7×10⁶ cell/ml

WBC=(6.44+3.97)×36.7/100=3.82×10⁶ cell/ml

Number of ISC and others=2.35×36.7/100=0.86×10⁶ cell/ml

Number of sperm cells: 36.7−(3.82+0.86)=32×10⁶ cell/ml

Sample 2:

Total cells count: 16.2×10⁶ cell/ml

Number of WBC: (11.97+0.37)×16.2/100=1.98×10⁶ cell/ml

Number of ISC and others=0.08×16.2/100=0.01×10⁶ cell/ml

Number of sperm cells=16.2−(1.98+0.01)=14.21×10⁶ cell/ml

Thus, the counting result for sample 1 is higher by 9.6% (32; 29.2) and for sample 2 is higher by 4.48% (14.21; 13.6) than the one obtained by subtracting only the auto-fluorescence background and aggregates events.

### Example 2

### Evaluation of a Low Count Semen Sample Using the Flow Cytometry Method of the Present Invention

Materials and Methods

Traditional sperm cell counting: performed as described herein above for Example 1.

Flow cytometry sperm cell counting: performed as described herein above for Example 1 except that the total count in tube (d) was read by setting the instrument to analyze the tube for three minutes (3) and not to be limited by reaching a specific number of events—thereby allowing screening of large volumes of sample, enabling detection of even a few number of sperm cells.

Results

Five semen samples were analyzed by the traditional method and defined as azoospermic (zero sperm cells). Using the Flowcytometric method of the present invention, a few sperm cells were noticed. Further investigation of those samples by the Routine method that included centrifugation of the sample and repeated counting (10 times) of aliquots of the sample pellet also revealed a few cells. The results are shown in the table below:

TABLE 3

| | Routine Count | FC Count | Further |
|---|---|---|---|
| Sample # | 10⁶ cell/ml | 10⁶ cell/ml | investigation - Routine |
| 1 | 0 | 0.0005 | Few cells |
| 2 | 0 | 0.00003 | One cell |
| 3 | 0 | 0.00004 | Two cells |
| 4 | 0 | 0.0006 | Few cells |
| 5 | 0 | 0 | 0 |

### Example 3

### Reproducibility of the Flowcytometric Method of the Present Invention

The reproducibility of the Flowcytometric based method of the present invention was evaluated by re-testing the same sample five times and comparing the results.

Results

Table 4 below summarizes the sperm cell count results of 11 samples. Table 5 below summarizes the WBC count results of the 11 samples. The low standard deviation indicates high reproducibility of Sperm and WBC count.

TABLE 4

| | | | | | | Standard |
|---|---|---|---|---|---|---|
| # | 1 | 2 | 3 | 4 | 5 | Deviation |
| 1 | 89.96 | 88.89 | 88.64 | 88.15 | 89.68 | 0.75 |
| 2 | 74.41 | 70.57 | 65.74 | 69.84 | 72.74 | 3.29 |
| 3 | 26.44 | 22.97 | 23.43 | 17.16 | 25.32 | 3.59 |
| 4 | 30.66 | 29.53 | 31.59 | 29.8 | 28.04 | 1.33 |
| 5 | 24.02 | 23.67 | 23.48 | 21.57 | 25.52 | 1.41 |
| 6 | 29.69 | 29.93 | 29.9 | 30.88 | 30.29 | 0.47 |
| 7 | 45.39 | 45.16 | 43.81 | 47.73 | 44.07 | 1.55 |
| 8 | 53.73 | 50.25 | 51.15 | 52.06 | 51.33 | 1.30 |
| 9 | 25.3 | 27.81 | 25.82 | 24.34 | 26.99 | 1.37 |
| 10 | 59.45 | 62.96 | 65.53 | 63.38 | 64.84 | 2.36 |
| 11 | 55.6 | 49.74 | 50.87 | 50.48 | 51.97 | 2.31 |

TABLE 5

| | | | | | | Standard |
|---|---|---|---|---|---|---|
| # | 1 | 2 | 3 | 4 | 5 | Deviation |
| 1 | 41.72 | 40.57 | 40.26 | 36.07 | 35.94 | 2.71 |
| 2 | 40.47 | 38.42 | 39.48 | 38.9 | 41.15 | 1.12 |
| 3 | 8.5 | 10.72 | 8.95 | 8.34 | 8.84 | 1.04 |
| 4 | 6.4 | 5.85 | 7.34 | 5.69 | 5.09 | 0.85 |
| 5 | 0.52 | 0.6 | 0.56 | 0.5 | 0.48 | 0.05 |
| 6 | 9.81 | 8.75 | 8.86 | 8.52 | 8.59 | 0.52 |
| 7 | 10.5 | 11.17 | 9.45 | 9.42 | 9.69 | 0.77 |
| 8 | 11.2 | 11.68 | 8.49 | 8.44 | 9.48 | 1.51 |
| 9 | 1.63 | 1.32 | 1.34 | 1.48 | 1.32 | 0.14 |
| 10 | 4.12 | 6.9 | 5.88 | 4.43 | 3.93 | 1.29 |
| 11 | 4.84 | 3.04 | 3.14 | 3 | 2.85 | 0.83 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

What is claimed is:

1. A flow cytometry method for quantifying an absolute number of mature sperm cells in a semen sample, the method comprising:
(a) determining a total particle number (T);
(b) determining a number of DNA comprising particles (D);
(c) determining a number of cells which are not mature sperm cells (B); and
(d) determining a background signal (A);
wherein steps (a) to (d) are not effected in a single aliquot of the semen sample, and whereas the number of mature sperm cells in the semen sample is equal [(T×D)/100]−(A+B), thereby quantifying the absolute number of mature sperm cells in the semen sample.

2. The method of claim 1, wherein said number of cells which are not mature sperm cells comprises a white blood cell number, and/or a number of cells of a substantially different size than said mature sperm cells.

3. The method of claim 1, wherein each of said aliquots comprise an identical volume.

4. The method of claim 1, wherein each of said background signal, said total particle number, said number of DNA comprising particles and said number of cells which are not mature sperm cells is determined in a separate aliquot.

5. The method of claim 2, wherein determining said white blood cell number is effected via a white blood cell specific antibody.

6. The method of claim 5, wherein said white blood cell specific antibody is anti-CD-45.

7. The method of claim 1, wherein step (a) is effected via fluorescent beads.

8. The method of claim 1, wherein determining said number of DNA comprising particles is effected via a DNA staining agent.

9. The method of claim 8, wherein said DNA staining agent is acridine orange or propidium iodide.

10. The method of claim 1, wherein the semen sample is a human semen sample.

11. The method of claim 1, wherein the semen sample is a bull semen sample.

12. A flow cytometry method for quantifying a relative number of mature sperm cells in a semen sample, the method comprising:
(a) determining a number of DNA comprising particles (D);
(b) determining a number of cells which are not mature sperm cells (B); and
(c) determining a background signal (A);
wherein steps (a)-(c) are not effected in a single aliquot of the semen sample, and whereas the number of mature sperm cells in the semen sample is equal to D−(A+B), thereby quantifying the relative number of mature sperm cells in the semen sample.

13. The method of claim 12, wherein said cells which are not mature sperm cells comprise white blood cells, and/or cells of a substantially different size than said mature sperm cells.

14. The method of claim 12, wherein each of said aliquots comprise an identical volume.

15. The method of claim 12, wherein each of said background signal, said number of DNA comprising particles and said number of cells which are not mature sperm cells is determined in a separate aliquot.

16. The method of claim 13, wherein determining said number of white blood cells is effected via a white blood cell specific antibody.

17. The method of claim 16, wherein said white blood cell specific antibody is anti-CD-45.

18. The method of claim 12, wherein determining said number of DNA comprising particles is effected via a DNA staining agent.

19. The method of claim 18, wherein said DNA staining agent is acridine orange or propidium iodide.

20. The method of claim 12, wherein the semen sample is a human semen sample.

21. The method of claim 12, wherein the semen sample is a bull semen sample.

22. A kit for determining a number of sperm cells in a semen sample, wherein the kit comprises:
(i) a white blood cell specific antibody;
(ii) a DNA staining agent;
(iii) a plurality of fluorescent beads; and
(iv) a plurality of tubes wherein each four of said tubes are packaged in a single packaging.

23. The kit of claim 22, wherein the white blood cell specific antibody is anti-CD-45.

24. The kit of claim 22, wherein said DNA staining agent is acridine orange or propidium iodide.
